(19) United States
(12) Patent Application Publication
(10) Pub. No.: US 2007/0207116 A1
(43) Pub. Date: Sep. 6, 2007

(54) Antioxidant compositions for the eye

(76) Inventor: David C. Brown, Fort Myers, FL (US)

Correspondence Address: BAKER BOTTS, LLP, 910 LOUISIANA, HOUSTON, TX, 77002-4995, US

(21) Appl. No.: 11/365,731

(22) Filed: Mar. 1, 2006

(51) Int. Cl. A61K 38/40; A61K 38/36; A61K 38/05; A61K 31/728; A61K 31/717; A61K 36/886

(52) U.S. Cl. 424/78.3; 424/744; 514/18; 514/171; 514/440; 514/419; 514/456; 514/474; 514/57; 424/78.37; 514/12; 514/690; 514/733; 514/54

(57) ABSTRACT

Ophthalmic compositions comprising at least one antioxidant agent chosen from a carotenoid, glutathione, reduced glutathione, glutathione enhancers, a lipoic acid, a flavonoid, an oleanoic acid, ascorbyl palmitate, aloe vera extract, an omega-6 fatty acid, melatonin, and vitamin E acetate.

### BACKGROUND

This invention relates to compositions useful in treatment of mammalian eyes. The compositions of the present invention may be used to treat, among other things, one or more of irritations of the eye, dryness of the eye, and the onset or progression of an eye disease.

Eye diseases threaten the vision of many people, and their treatment often is costly. Eye irritations are also problematic, among other things, because they cause great discomfort and can lead to secondary eye problems, such as infection.

A number of eye diseases and irritations are associated with free radicals. A free radical is a molecular species having a single unpaired electron available in an outer orbital. Free radicals can initiate chain reactions in cells and body fluids that damage organic molecules, including biomolecules (e.g., DNA, lipids, and proteins). Free radicals are formed endogenously (e.g., by cellular metabolism, inflammation by immune cells, and the like) and exogenously (e.g., by radiation, pharmaceuticals, hydrogen peroxide, toxic chemicals, smoke, alcohol, oxidized polyunsaturated fats, and the like). For example, the eye is exposed to light whenever it is open and light entering the eye may generate a superoxide radical in the eye. Once generated, the superoxide radical may degenerate into other free radicals such as hydrogen peroxide and hydroxide radicals.

The damage caused by free radicals often is referred to as “oxidative stress.” Oxidative stress—the cytotoxic consequences of a mismatch between the production of free radicals and the ability of a cell to defend against them—may be caused by an increase in the formation of free radicals, or by a decrease in scavenging of free radicals or repair of free-radical-modified macromolecules, or both. Oxidative stress is associated with a variety of ocular disorders such as cataracts, glaucoma, diabetic retinopathy, and macular degeneration. For example, in the case of cataracts (a loss of transparency of the lens), free radicals may oxidize the proteins of the lens, which may damage the protein structure of the lens.

Certain molecules, termed antioxidants, are capable of scavenging free radicals and subsequently protecting cells from damage due to oxidative stress. Antioxidants protect cells from free radicals by inhibiting free radical formation, intercepting free radicals, and repairing free-radical-induced injury. However, delivery of antioxidants to an eye is difficult. When administered orally, antioxidants may have, among other things, poor gastrointestinal absorption, a high dosage requirement, and lower bioavailability. Methods and compositions are needed for providing antioxidants in efficacious amounts directly to an eye.

### SUMMARY

This invention relates to compositions useful in treatment of mammalian eyes. The compositions of the present invention may be used to treat, among other things, one or more of irritations of the eye, dryness of the eye, and the onset or progression of an eye disease.

In certain embodiments, the present invention provides compositions comprising at least one antioxidant agent chosen from a carotenoid, glutathione, reduced glutathione, glutathione enhancers, a lipoic acid, a flavonoid, an oleanoic acid, ascorbyl palmitate, aloe vera extract, an omega-6 fatty acid, melatonin, and vitamin E acetate. The compositions of the present invention may be used in the methods of the present invention to treat an eye of a mammal.

The compositions of the present invention have antioxidant activity. The term “antioxidant activity” refers to an inhibitory effect on biological oxidative processes involving free radicals or singlet oxygen. In addition to antioxidant activity, certain embodiments of the compositions of the present invention also may have activity as one or more of a lubricant, a nitric oxide synthase inhibitor, an anti-inflammatory, a neuroprotectant, a bacteriocide, and a bacteriostat.

The compositions of the present invention may be used as either or both a common eye treatment (e.g., an eye drop) and a specific drug in the treatment of an eye disease. If used prior to the onset of an eye disease, the compositions assist in preserving the integrity of the eye's antioxidant defense systems in order to prevent, or at least greatly delay, the onset of an eye disease, such as cataracts. If used after an eye disease is detected, the compositions may act to restore the antioxidant defense system and thereby prevent, or greatly slow, further progression of an eye disease.

The compositions of the present invention may be in a variety of forms, such as the form of a solution, a suspension, an emulsion, a film, a spray, or the like. The viscosity of the compositions may be increased to increase retention time in the eye, reduce drainage rates, or increase ocular bioavailability. The compositions of the present invention offer a significant advantage over oral administration of antioxidant agents, among other things, by overcoming the difficulty of poor gastrointestinal absorption, by using a lower dosage than would be required by oral administration, by allowing more of the active agents to provide therapeutic relief, and by lessening systemic absorption.

The features and advantages of the present invention will be readily apparent to those skilled in the art upon a reading of the description of the embodiments that follows.

### DESCRIPTION

This invention relates to compositions useful in treatment of mammalian eyes. The compositions of the present invention may be used to treat, among other things, one or more of irritations of the eye, dryness of the eye, and the onset or progression of an eye disease. The present invention provides compositions that may be used to maintain eye health as well as treat and prevent eye diseases. As referred to herein, the term “eye diseases” will be understood to mean eye disorders including, but not limited to, cataract, open-angle primary glaucoma, corneal disorders, presbyopia, computer vision syndrome, eye strain, ocular inflammation, blurred vision, dry eye syndrome, retinal diseases, vitreous opacities and lesions, complications of diabetes mellitus, and macular and other retinal diseases.

The compositions of the present invention comprise at least one antioxidant agent chosen from carotenoids, glutathione and glutathione enhancers, lipoic acids, flavonoids, oleanoic acids, ascorbyl palmitate, aloe vera extract, omega-6 fatty acids, melatonin, and vitamin E acetate. The compositions of the present invention also may comprise a lubricating agent, as well as other components of therapeutic benefit or useful to form a composition for delivery to the eye.

The concentration of the antioxidant agents in the compositions of the present invention may vary depending on a variety of factors, such as, for example, type of antioxidant agent used, bioavailability, potency, and the like. In general, the antioxidant agents should be present in the compositions of the present invention in the range of from about 0.001% to about 10% by weight of the composition, and may be suspended or dissolved. Antioxidant agents are available commercially from a variety of suppliers, including distributors, such as TSN Labs, Inc., Midvale, Utah and Amira, Alachua, Fla., as well as manufacturers, such as AstaCarotene AB, Gustravsberg, Sweden and Technical Sourcing International, Inc., Missoula, Mont.

One example of antioxidant agents suitable for use in the compositions of the present invention includes, among other things, carotenoids. In certain embodiments, carotenoids may be present in the compositions of the present invention in an amount in the range of from about 0.05% to about 2% by weight of the composition. Examples of suitable carotenoids include, but are not limited to, lutein and zeaxanthin, which are naturally found in normal mammalian eyes. Although these carotenoids are naturally present in mammalian eyes, the amount present typically decreases as the mammal's age increases. Zeaxanthin protects the retina by absorbing ultraviolet light, blocks the activity of peroxide radicals, inhibits low-density lipoprotein (LDL) oxidation, and consequently protects cell membranes from this and other free radical damage. Lutein filters out blue light from the retina and reduces oxidative stress or free radical damage in the macula. Sulforaphane is another example of carotenoid suitable for use as an antioxidant agent in the compositions of the present invention. Sulforaphane can convert to glucosinolates in the human body, which may induce production of antioxidant-detoxifying enzymes such as glutatione-5-transferase and UDP-glucuronosyl transferase. Examples of other carotenoids that may be used as antioxidant agents in the compositions of the present invention include, but are not limited to, astaxanthin, astaxanthene, α-carotene, β-carotene, cantaxanthin, luteulin, lycopene, phystoene, fucoxanthan, periodinin, and xanthophylls.

Other examples of antioxidant agents suitable for use in the compositions of the present invention include, among other things, glutathione, reduced glutathione, and glutathione enhancers. Glutathione, reduced glutathione, and glutathione enhancers each may be present in the compositions of the present invention in an amount in the range of from about 0.05% to about 2% by weight of the composition. When reduced, glutathione scavenges, or neutralizes, a free radical to a less toxic or non-toxic molecule; glutathione then becomes “oxidized,” most often to the glutathione disulfide anion radical. High levels of glutathione are found in the lens of the eye, and glutathione levels decrease as cataracts form. Examples of suitable glutathione enhancers include, among other things, L-cysteine, pyridoxine, and riboflavin. Other examples of suitable glutathione enhancers include, among other things, glutathione precursors, such as N-acetylcysteine.

Other examples of antioxidant agents suitable for use in the compositions of the present invention include, among other things, lipoic acids (e.g., α-lipoic acid, dihydrolipoic acid, and isolipoic acid). Besides lipoic acid's ability to inhibit free radicals, it is involved with regenerating other antioxidants that may protect the eye. Treatment with lipoic acid also may enhance the activities of other antioxidant scavenging enzymes such as glutathione peroxidase, catalase, and ascorbate free radical reductase. In addition to having antioxidant properties, lipoic acid has antiinflammatory and analgesic properties. Lipoic acids may be present in the compositions of the present invention in an amount in the range of from about 0.05% to about 2% by weight of the composition.

Other examples of antioxidant agents suitable for use in the compositions of the present invention include, among other things, flavonoids. Flavonoids are compounds that are present in a variety of plants and include phenolic compounds (e.g., proanthocyanins, anthocyanins, flavanoids, flavones, flavanones, flavonols, flavans, isoflavones, catechins, epicatechins, resveratrol, and phenolic acids) and monoterpenes (e.g., limonene). Flavonoids readily scavenge superoxide and hydroxyl free radicals and can inhibit lipid peroxidation, which may occur when a mammalian eye is exposed to ultraviolet radiation. In certain embodiments, flavonoids may be present in the compositions of the present invention in an amount in the range of from about 0.05% to about 2% by weight of the composition. Examples of suitable flavonoids include, but are not limited to, proanthocyanins. Proanthocyanins are phenolic polymers built from catechin or epicatechin monomer units and include, for example, catechin, epicatechin, catechin gallate, epicatechin gallate, gallocatechin gallate, and epigallocatechin gallate. Generally, the proanthocyanins are from two to seven catechin units long. Longer oligomers, and the monomeric catechin and epicatechin units, also have oxygen-scavenging capability. Proanthocyanins are found naturally in a variety of botanicals, such as grape seeds, pine bark, blackjack oak, horse chestnut, witch hazel, and hawthorn. Proanthocyanins are further found in apples, berries, barley, bean hulls, chocolate, rhubarb, rose hips, and sorghum. Synthetic analogs of the botanical extracts also exist and one skilled in the art, with the benefit of this disclosure, will recognize that such synthetic analogs may also be utilized in the present invention. In addition to having antioxidant properties, proanthocyanins also may be a neuronal cell protectant against cytotoxicity, and have the ability to regenerate the ascorbyl radical and protect endogenous vitamin E and glutathione from oxidative stress. Another example of a suitable flavonoid is resveratrol (3,4′,5-trihydroxystilbene). Resveratrol and its glucoside, cis- and trans-forms occur naturally in a number of plant families including Vitaceae. These resveratrols possess many biological activities including antioxidant activity and antiinflammatory properties. Other examples of suitable flavonoids include, but are not limited to, quercetin, rutin, genistein, citrus bioflavonoids (e.g., narigingin and flavone glycosides such as hesperidin), ellagic acid, and hydroxytyrosol.

Other examples of antioxidant agents suitable for use in the compositions of the present invention include, among other things, oleanoic acids. Oleanoic acids are antioxidants commonly found in plants. Oleanoic acids are capable of inhibiting free radicals. In certain embodiments, oleanoic acids may be present in the compositions of the present invention in an amount in the range of from about 0.05% to about 2% by weight of the composition.

Another example of an antioxidant agent suitable for use in the compositions of the present invention includes, among other things, ascorbyl palmitate. Ascorbyl palmitate is a synthetic ester of vitamin C that is fat-soluble, has a neutral pH, and is stable. Ascorbyl palmitate also may have antiinflammatory activity within a cell, such as a cell of a mammalian eye. In certain embodiments, ascorbyl palmitate may be present in the compositions of the present invention in an amount in the range of from about 0.05% to about 2% by weight of the composition.

Another example of an antioxidant agent suitable for use in the compositions of the present invention includes, among other things, aloe vera extract. Aloe vera extract is the mucilaginous substance produced by the parenchymal cells of the aloe vera plant, which is known to have antioxidant, anti-inflammatory, antibacterial, and antiviral properties. Aloe vera extract is a complex mixture of components that includes, among other things, mono- and polysaccharides (e.g., acemannan), glycoproteins (e.g., alprogen, C-glucosyl chromone), lignan, salicylic acid, saponins, sterols, triterpenoids, glutathione peroxidase, superoxide dismutase isozymes, as well as vitamins and minerals. Aloe vera extract also may induce expression of the antioxidant protein, metallothionein, as well as scavenge hydroxyl radicals and prevent suppression of superoxide dismutase and glutathione peroxidase. In certain embodiments, aloe vera extract may be present in the compositions of the present invention in an amount in the range of from about 0.05% to about 2% by weight of the composition.

Other antioxidant agents suitable for use in the compositions of the present invention include, among other things, omega-6 fatty acids (e.g., γ-linolenic acid (GLA) and linoleic acid (LA)) and omega-3 fatty acids (e.g., eicosopentaenoic acid (EPA) and docosahexaenoic acid (DHA)). In certain embodiments, these fatty acids may be present in the compositions of the present invention, alone or in combination, in an amount in the range of from about 0.05% to about 2% by weight of the composition.

Other antioxidant agents suitable for use in the compositions of the present invention include, among other things, melatonin and vitamin E acetate. In certain embodiments, melatonin may be used in the compositions of the present invention in an amount in the range of from about 0.05% to about 2% by weight of the composition. In other embodiments, vitamin E acetate may be used in the compositions of the present invention in an amount in the range of from about 0.05% to about 1.5% by weight of the composition.

In certain embodiments, two or more antioxidant agents may be chosen and combined so as to have a synergistic antioxidant activity. Synergy of antioxidant agent combinations may be measured using an antioxidant activity assay known in the art, for example, the Total Oxyradical Scavenging Capacity (TOSC) Assay.

In certain embodiments, the compositions of the present invention also may comprise a lubricating agent, which may, among other things, treat eye irritation, dryness, and provide lubrication for the eyes. Any known lubricating agent suitable for use in an ophthalmic preparation may be used in the compositions of the present invention. The lubricating agent should be inert to other composition components. Accordingly, the lubricating agent may be chosen based on the antioxidant agent chosen. Examples of suitable lubricating agents include, but are not limited to, cellulose derivatives (such as sodium carboxymethyl cellulose and hydroxypropyl methylcellulose), glycerin, polyvinyl alcohol, polyvinylpyrrolidone, povidone, propylene glycol, mineral oil, and the like. Lubricating agents are available from sources well-known in the industry.

When present, the lubricating agent may be used in the compositions of the present invention an amount in the range of from about 0.01% to about 3% by weight of the composition. For example, in certain embodiments in which the lubricating agent is hydroxypropyl methylcellulose, it may be used in the compositions of the present invention in an amount in the range of from about 0.2% to about 0.3% by weight of the composition. In other embodiments in which the lubricating agent is carboxymethylcellulose, it may be used in the compositions in an amount in the range of from about 0.2% to about 1.5% by weight of the composition. In other embodiments in which the lubricating agent is polyvinyl alcohol, it may be used in the compositions in an amount in the range of from about 0.5% to about 2% by weight of the composition.

Other suitable lubricating agents include lactoferrin and albumin. These may be especially suited for the treatment of dry-eye syndrome. Typically, these ingredients are used in the dosages in which they are present in the natural tear fluid. For example, a typical dose of human lactoferrin may be about 1.7 mg/mL.

The compositions of the present invention also may comprise other components useful to form an ophthalmic preparation. Such components include a vehicle (e.g., water), buffers, organic carriers, inorganic carriers, emulsifiers, wetting agents, and the like. In certain embodiments, the compositions of the present invention also may comprise other components, including anti-inflammatory agents, penetration enhancers (e.g., methyl-sulfonyl-methane), nerve sheath protectants (e.g., propylene glycol), nitric oxide synthase inhibitors (e.g., L-arginine), hyaluronic acid, and collagen.

Examples of vehicles include water, water mixtures of lower alkanols, vegetable oils, polyalkylene glycols, petroleum-based jelly, ethylcellulose, ethyl oleate, carboxymethylcellulose, polyvinylpyrrolidone, and isopropyl myristrate. Some antioxidant agents are poorly absorbed by the mammalian eye and are not administrable as drops or spray; ointments or semisolid aqueous gel bases (e.g., carbomer) may thus be used.

In general, the compositions of the present invention should have an osmotic pressure sufficient to approximate the osmotic pressure of the fluids naturally found in the mammalian eye. If necessary, the osmotic pressure can be adjusted by using appropriate amounts of physiologically and opthalmologically acceptable salts or excipients. For example, sodium chloride may be added to the compositions of the present invention to approximate fluids naturally found in the mammalian eye. When included, sodium chloride typically is used in amounts ranging from about 0.01% to about 1% by weight of the composition. Equivalent amounts of one or more salts made up of cations (e.g., potassium and ammonium) and anions (e.g., chloride, citrate, ascorbate, borate, phosphate, bicarbonate, sulfate, thiosulfate, bisulfate, sodium bisulfate, and ammonium sulfate) may also be used in addition to or instead of sodium chloride to achieve osmolarities within the above-stated range. Sugars like mannitol, dextrose, glucose, or other polyols also may be added to adjust osmolarity.

The compositions of the present invention also may comprise a buffering agent to, among other things, control pH and to prevent pH drift under storage conditions. Any pharmaceutically acceptable buffering agent may be utilized. Suitable buffering agents are known in the art and may include one or more of zinc sulfate, boric acid, sodium borate, potassium (e.g., potassium bicarbonate), sodium phosphate, sodium acetate, and sodium citrate. When present, the particular concentration will vary, depending on the agent employed. In general, however, the buffering agent should be used in an amount sufficient to maintain a target pH in the range of from about 6.0 to about 8.0. An additional benefit of including zinc sulfate as a buffering agent is its role as a cofactor for antioxidant scavenging enzymes. An additional benefit from including potassium as a buffering agent is that potassium may assist in the expulsion of sodium from the lens and eye, thereby reducing dryness and irritation of the eye.

The compositions of the present invention also may include a preservative. Any known preservative suitable for ophthalmic use may be used. For example, the preservative may be benzalkonium chloride and other quaternary ammonium preservative agents, phenylmercuric salts, sorbic acid, chlorobutanol, disodium edetate, ethylenediaminetetraacetic acid (EDTA), thimerosal, methyl- and propylparaben, benzyl alcohol, and phenyl ethanol. When present, the amount of preservative used may depend on the particular preservative chosen. In certain embodiments, the preservative may be present in the compositions of the present invention in an amount of from about 0.001% to about 1% by weight of the composition. In certain embodiments, the optional preservative should be chosen to minimize any reduction in the storage stability of the components present in the composition, or to minimize any adverse interactions with these components, or both. Examples of such a preservative are the systems disclosed in U.S. Pat. Nos. 5,576,028 and 5,607,698, the relevant disclosures of which are incorporated herein by reference. These systems use a low amount of hydrogen peroxide, or a peroxide source, as a preservative in combination with a peroxy stabilizer, such as a phosphonic acid (e.g., diethylene triamine penta(methylene-phosphonic acid).

In some embodiments, the compositions of the present invention may comprise a sustained release topical ophthalmic delivery system to release the antioxidant agent to the eye. Such a system is commercially available under the trade name DuraSite® and available from InSite Vision Inc., Alameda, Calif.

One example of a formulation of a composition of the present invention is a composition comprising the components in Table 1.

TABLE 1

| Amount (%) | Components |
|---|---|
| 0.4 | Aloe vera extract |
| 0.5 | Zinc sulfate heptahydrate |
| 0.01 | Benzalkonium chloride |
| 99.09 | Other components such as, for |
| | example, sodium citrate, sodium |
| | chloride, citric acid, and water |

Another example of a formulation of a composition of the present invention is a composition comprising the components in Table 2.

TABLE 2

| Amount (%) | Components |
|---|---|
| 1 | Resveratrol |
| 0.3 | Glycerin |
| 1 | Propylene glycol |
| 0.01 | Benzalkonium chloride |
| 0.025 | EDTA |
| 97.665 | Other components such as, for example, |
| | boric acid, potassium chloride, sodium |
| | chloride, sodium borate, sodium |
| | hydroxide (to adjust pH), and water |

Another example of a formulation of a composition of the present invention is a composition comprising the components in Table 3.

TABLE 3

| Amount (%) | Components |
|---|---|
| 0.5 | Lutein |
| 0.2 | Zeaxanthin |
| 0.2 | Hydroxypropyl methylcellulose |
| 99.1 | Other components such as, for |
| | example, boric acid, calcium chloride, |
| | sodium perborate, phosphoric acid, |
| | potassium chloride, sodium chloride, |
| | and water |

Another example of a formulation of a composition of the present invention is a composition comprising the components in Table 4.

TABLE 4

| Amount (%) | Components |
|---|---|
| 0.4 | Aloe vera extract (freeze dried powder) |
| 1 | Propylene glycol |
| 0.3 | Glycerin |
| 0.01 | Benzalkonium chloride |
| 0.75 | Boric acid |
| 0.025 | Disodium edetate |
| 97.515 | other components such as, for example, |
| | potassium chloride, sodium chloride, |
| | sodium borate, sodium hydroxide (to |
| | adjust pH), and water |

In treating an eye, the compositions of the present invention may be added to the eye in an amount of from, for example, 1 to 8 drops per day. A drop has an average volume of about 50 microliters. The applications are preferably distributed evenly over the course of a day so that the applications occur from, for example, 1 to 4 times per day.

Therefore, the present invention is well adapted to carry out the objects and attain the ends and advantages mentioned as well as those that are inherent therein. While numerous changes may be made by those skilled in the art, such changes are encompassed within the spirit of this invention as defined by the appended claims.

What I claim is:

1. An ophthalmic composition comprising at least one antioxidant agent chosen from a carotenoid, glutathione, reduced glutathione, glutathione enhancers, a lipoic acid, a flavonoid, an oleanoic acid, ascorbyl palmitate, aloe vera extract, an omega-6 fatty acid, melatonin, and vitamin E acetate.

2. The composition of claim 1, further comprising a lubricating agent.

3. The composition of claim 1, further comprising at least one lubricating agent chosen from a cellulose derivative, sodium carboxymethyl cellulose, hydroxypropyl methylcellulose, glycerin, polyvinyl alcohol, polyvinylpyrrolidone, povidone, propylene glycol, mineral oil, lactoferrin, and albumin.

4. The composition of claim 1, further comprising at least one additional component chosen from a vehicle, a buffer, an organic carrier, an inorganic carrier, an emulsifier, a wetting agent, an anti-inflammatory agent, a penetration enhancer, a nerve sheath protectant, a nitric oxide synthase inhibitor, a hyaluronic acid, a collagen, and a preservative.

5. The composition of claim 1, further comprising 0.4% aloe vera extract, 0.5% zinc sulfate heptahydrate, and 0.01% benzalkonium chloride.

6. The composition of claim 1, further comprising 1% resveratrol, 0.3% glycerin, 1% propylene glycol, 0.01% benzalkonium chloride, and 0.025% EDTA.

7. The composition of claim 1, further comprising 0.5% lutein, 0.2% zeaxanthin, and 0.2% hydroxypropyl methylcellulose.

8. The composition of claim 1, further comprising 0.4% aloe vera extract, 1% propylene glycol, 0.3% glycerin, 0.01% benzalkonium chloride, 0.75% benzalkonium chloride, 0.75% boric acid, 0.025% disodium edetate.

9. A method for treating an eye comprising administering to the eye an ophthalmic composition comprising at least one antioxidant agent chosen from a carotenoid, glutathione, reduced glutathione, glutathione enhancers, a lipoic acid, a flavonoid, an oleanoic acid, ascorbyl palmitate, aloe vera extract, an omega-6 fatty acid, melatonin, and vitamin E acetate.

10. The method of claim 9, wherein the ophthalmic composition further comprises a lubricating agent.

11. The method of claim 9, wherein the ophthalmic composition further comprises at least one lubricating agent chosen from a cellulose derivative, sodium carboxymethyl cellulose, hydroxypropyl methylcellulose, glycerin, polyvinyl alcohol, polyvinylpyrrolidone, povidone, propylene glycol, mineral oil, lactoferrin, and albumin.

12. The method of claim 9, wherein the ophthalmic composition further comprises at least one additional component chosen from a vehicle, a buffer, an organic carrier, an inorganic carrier, an emulsifier, a wetting agent, an anti-inflammatory agent, a penetration enhancer, a nerve sheath protectant, a nitric oxide synthase inhibitor, a hyaluronic acid, a collagen, and a preservative.

13. The method of claim 9, wherein the ophthalmic composition further comprises 0.4% aloe vera extract, 0.5% zinc sulfate heptahydrate, and 0.01% benzalkonium chloride.

14. The method of claim 9, wherein the ophthalmic composition further comprises 1% resveratrol, 0.3% glycerin, 1% propylene glycol, 0.01% benzalkonium chloride, and 0.025% EDTA.

15. The method of claim 9, wherein the ophthalmic composition further comprises 0.5% lutein, 0.2% zeaxanthin, and 0.2% hydroxypropyl methylcellulose.

16. The method of claim 9, wherein the ophthalmic composition further comprises 0.4% aloe vera extract, 1% propylene glycol, 0.3% glycerin, 0.01% benzalkonium chloride, 0.75% benzalkonium chloride, 0.75% boric acid, 0.025% disodium edetate.
